(19) United States
(12) Patent Application Publication
(10) Pub. No.: US 2025/0325354 A1
(43) Pub. Date: Oct. 23, 2025

(54) Protocol for Preparing and Fitting Prosthetics Using Pre-surgical Fiducial Markers and Registration Images Formed Thereby

(71) Applicant: Smile In One System, LLC, Boca Raton, FL (US)

(72) Inventors: Johnnie Orfanidis, Jacksonville, FL (US); Nilo A. Hernandez, JR., Dadeville, AL (US)

(21) Appl. No.: 19/199,327

(22) Filed: May 5, 2025

Related U.S. Application Data
(63) Continuation of application No. PCT/US23/36812, filed on Nov. 3, 2023.
(60) Provisional application No. 63/422,415, filed on Nov. 3, 2022.

(51) Int. Cl. A61C 13/00; A61B 1/24; A61B 6/51; G06T 7/00; G16H 20/40

(52) U.S. Cl. CPC A61C 13/0004; A61B 1/24; A61B 6/51; G06T 7/0016; G16H 20/40; G06T 2207/20212; G06T 2207/30036; G06T 2207/30052; G06T 2207/30201; G06T 2207/30204

(57) ABSTRACT

A protocol for preparing and fitting a prosthetic in a prosthetic field of a patient using pre-surgical fiducial markers comprises obtaining pre-surgical physiologic data of the patient sufficient to create a base patient image of the prosthetic field; determining fiducial marker placement and placing fiducial markers in the determined fiducial marker placement in physiologic structure within the prosthetic field which is unchanged by planned surgery; optionally obtaining pre-surgical physiologic data of the patient with the fiducial markers in place sufficient to create a base patient image of the prosthetic field with fiducial markers; performing surgery; obtaining post-surgical physiologic data of the patient with the fiducial markers sufficient to create a post-surgical patient image of the prosthetic field with fiducial markers; manufacturing and fitting a final prosthetic based upon post-surgical patient image of the prosthetic field with fiducial markers. Registration images useful for the protocol are disclosed.

### RELATED APPLICATIONS

This application is a continuation of International Application Serial Number PCT/US2023/036812 which filed Nov. 3, 2023 and published May 10, 2024 as publication number WO 2024/097419. International Application Serial Number PCT/US2023/036812 claims the benefit of U.S. Provisional Patent Application Ser. No. 63/422,415 filed Nov. 3, 2022. This publication and these applications are incorporated herein by reference in its entirety.

### BACKGROUND OF THE INVENTION

### 1. Field of the Invention

This technology generally relates to a protocol for preparing and fitting prosthetics, particularly oral, orthopedic and ophthalmological prosthetics, using pre-surgical fiducial markers and registration images formed thereby.

### 2. Background Information

A prosthetic within the meaning of this application is an artificial device that replaces a missing body part, which may be lost through trauma, disease, or a condition present at birth such as a congenital disorder. A prosthetic field within the meaning of this application is the area within the body, including related or adjacent tissue within which a prosthetic fits and operates, analogous roughly to an operative field in surgery. Prostheses are intended to restore the normal functions of the missing body part. The present invention is described in connection with oral prosthetics, but can have broader application to fitting of any prosthetics associated with surgically altered physiologic parameters and is also particularly useful for orthopedic and ophthalmological prosthetics.

An oral prosthetic, also called a dental prosthesis, is an intraoral prosthetic used to restore/reconstruct intraoral defects such as missing teeth, missing parts of teeth, and missing soft or hard structures of the jaw or palate. The prosthetic field for oral prosthetics is the oral cavity and associated body structures. Prosthodontics is the dental specialty that focuses on dental prostheses. Such prostheses are used to rehabilitate mastication (chewing), improve aesthetics, and aid speech. A dental prosthesis may be held in place by connecting to teeth or dental implants, by suction, or by being held passively by surrounding muscles. Like other types of prostheses, they can either be fixed permanently or removable; fixed prosthodontics and removable dentures are made in many variations. Permanently fixed dental prostheses use dental adhesive or screws, to attach to teeth or dental implants. Removal prostheses may use friction against parallel hard surfaces and undercuts of adjacent teeth or dental implants, suction using the mucous retention (with or without aid from denture adhesives), and by exploiting the surrounding muscles and anatomical contours of the jaw to passively hold in place.

Dentures (also known as false teeth) are a common oral prosthetic constructed to replace missing teeth, and are supported by the surrounding soft and hard tissues of the oral cavity. Conventional dentures are removable (removable partial denture or complete denture). However, there are many denture designs, some which rely on bonding or clasping onto teeth or dental implants (fixed prosthodontics). There are two main categories of dentures, the distinction being whether they are used to replace missing teeth on the mandibular arch or on the maxillary arch.

As early as the 7th century BC, Etruscans in northern Italy made partial dentures out of human or other animal teeth fastened together with gold bands. The Romans had likely borrowed this technique by the 5th century BC. Wooden full dentures were invented in Japan around the early 16th century. In 1728, Pierre Fauchard described the construction of dentures using a metal frame and teeth sculpted from animal bone. The first porcelain dentures were made around 1770 by Alexis Duchâteau.

In 1791, the first British patent was granted to Nicholas Dubois De Chemant, previous assistant to Duchateau, for “a composition for the purpose of making of artificial teeth either single double or in rows or in complete sets, and also springs for fastening or affixing the same in a more easy and effectual manner than any hitherto discovered which said teeth may be made of any shade or colour, which they will retain for any length of time and will consequently more perfectly resemble the natural teeth.” He began selling his wares in 1792, with most of his porcelain paste supplied by the company Wedgwood.

17th century London's Peter de la Roche is believed to be one of the first ‘operators for the teeth’, men who advertised themselves as specialists in dental work. They were often professional goldsmiths, ivory turners or students of barber-surgeons. In 1820, Samuel Stockton, a goldsmith by trade, began manufacturing high-quality porcelain dentures mounted on 18-carat gold plates. Later dentures from the 1850s on were made of Vulcanite, a form of hardened rubber into which porcelain teeth were set. In the 20^{th} century, acrylic resin and other plastics were used.

Dental impressions have long been used to form dentures for patients. In the 21^{st} century digital dental impressions have become more standard. An intraoral scanner is a device, often shaped like a wand or large pen, which captures 3D digital dental impressions and uses the data to produce a highly detailed image of the prosthetic field, or patient's mouth. That image can be viewed on a computer screen or used to fabricate dental restorations. The scan is done in a fraction of the time compared to traditional physical impressions and today's intraoral scanners produce high-speed, high-precision results.

Even with the advancements in dental procedures, instruments, machining, and materials, an elusive and unsolved problem remains, namely the “precision” fitment of oral prosthetics. Regardless of the procedures used, existing placement methodologies for oral prosthetics have a significant and often unacceptable margin of error in the intraoral alignment of the oral prosthesis. This margin of error ultimately translates to poor fitting oral prosthetics, and sometimes less than ideal patient comfort.

One technology developed to assist in dental implant positioning in the X-GUIDE system from X-Nav Technologies. The X-Guide system uses a removable marker in the form of molded clip pressed into a 2-3 tooth impression, removed and allowed to harden. The hardened clips are placed over the teeth before a CBCT scan to create landmarks for guiding robotic surgical tools. The system however cannot address changes in the topography of a patient's mouth following surgical procedures.

Another technology developed to assist in dental implant positioning and dental surgery in general is the in the YOMI surgical robot from Neocis. The YOMI surgical robot uses a fiducial marker coupled to the patient's existing dentation the day of procedures for registration of the robot (and patient and tool virtual models) to the patient. The fiducial markers are replaced with a patient tracking arm to track patient movement and adjust robot (and patient model) with patient movement during procedure. Again the system however cannot address changes in the topography of a patient's mouth following surgical procedures.

One issue is that the topography of a patient's mouth pre and post tooth extraction changes significantly and on multiple planes. There are three factors that challenge the clinician in determining the ideal design and placement of the next, or final prosthesis in their treatment process. First is that after the extraction of teeth, the dentist loses reference points (i.e., landmarks) that could otherwise be used as a guide for the future prosthetic design and placement. Further, after the extraction of teeth, the gingiva (gum tissue) no longer has the integrity and support that it had prior to extraction. The removal of teeth leaves holes in the gingiva which distorts the gingiva as it reestablishes to the new environment. The soft tissue collapses around the socket which changes the geometry (shape and size) of the upper and lower gingiva. Additionally, when the teeth are extracted, they are removed from both the gingiva and the underlying bone. The remaining bone structure resorbs, and, in the process, there is a loss or reduction of bone which can range from 2-20 mm. This loss of structure further changes the topography of the gingiva. The combination of these factors when removing teeth has challenged dental professionals in providing a system that renders a perfect prosthesis fitment.

Regarding prior art, InstaRisa Technologies, LLC of Fresno California discloses, in U.S. Pat. Pub. 2022/016478, a “digital workflow process and associated devices and tools for providing dental restorations and, more particularly, to precise and efficient reconstruction and replacement of teeth using novel digital workflows and improved dental scan bodies, abutments and other dental devices and tools to efficiently achieve a more precise fit and optimal form and function.”

There is a need to improve preparing and fitting prosthetics, particularly oral prosthetics.

### SUMMARY OF THE INVENTION

The various embodiments and examples of the present invention as presented herein are understood to be illustrative of the present invention and not restrictive thereof and are non-limiting with respect to the scope of the invention.

One aspect of the present invention provides preparing and fitting prosthetics, particularly oral prosthetics. A protocol for preparing and fitting a prosthetic in a prosthetic field of a patient using pre-surgical fiducial markers according to one aspect of the present invention comprises the steps of: obtaining pre-surgical physiologic data of the patient in a prosthetic field sufficient to create a base patient image of the prosthetic field; determining fiducial marker placement in physiologic structure within the prosthetic field of the patient which is to be unchanged by a planned surgery; placing fiducial markers in the determined fiducial marker placement in physiologic structure within the prosthetic field which is to be unchanged by a planned surgery; optionally obtaining pre-surgical physiologic data of the patient in a prosthetic field with the fiducial markers in place sufficient to create a base patient image of the prosthetic field with fiducial markers; performing surgery altering physiologic structure of the patient within the prosthetic field; obtaining post-surgical physiologic data of the patient in a prosthetic field with the fiducial markers in place sufficient to create a post-surgical patient image of the prosthetic field with fiducial markers; manufacturing a final prosthetic based upon post-surgical patient image of the prosthetic field with fiducial markers; and fitting the final prosthetic in the prosthetic field based upon post-surgical patient image of the prosthetic field with fiducial markers.

One aspect of the present invention provides registration images used for preparing and fitting a prosthetic in a prosthetic field of a patient using pre-surgical fiducial markers, the registration images being a composite of comprising: a base patient image of a prosthetic field of the patient obtained with pre-surgical physiologic data of the patient in the prosthetic field; a pre-surgical base patient image of the prosthetic field with fiducial markers, wherein fiducial markers in the determined fiducial marker placement in physiologic structure within the prosthetic field which is to be unchanged by a planned surgery, and the pre-surgical base patient image of the prosthetic field with fiducial markers is obtained with physiologic data of the patient in a prosthetic field with the fiducial markers in place; and a post-surgical patient image of the prosthetic field with fiducial markers, whereby manufacturing of a final prosthetic is based upon post-surgical patient image of the prosthetic field with fiducial markers and fitting the final prosthetic in the prosthetic field is based upon post-surgical patient image of the prosthetic field with fiducial markers.

These and other advantages of the present invention are described below in connection with the attached figures in which like reference numerals represent like elements throughout.

### BRIEF DESCRIPTION OF THE FIGURES

FIG. 1 is a schematic view of the protocol for preparing and fitting a prosthetic in a prosthetic field of a patient using pre-surgical fiducial markers according to one aspect of the present invention; and

FIGS. 2A-D are front elevational, side elevation, top plan and perspective views of a fiducial marker used in the protocol of the present invention

### BRIEF DESCRIPTION OF THE PREFERRED EMBODIMENT

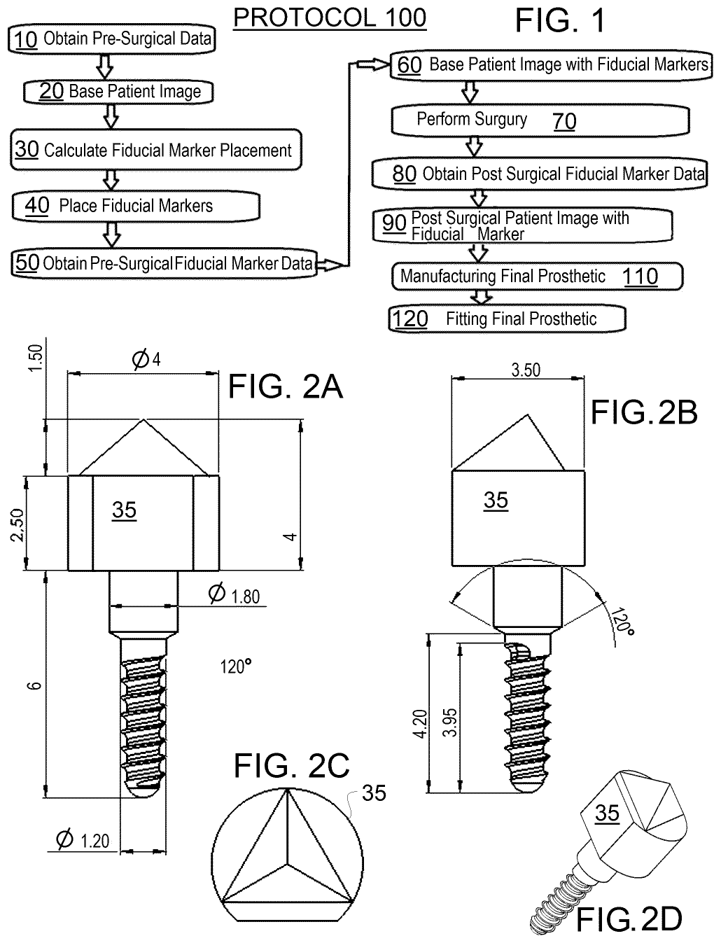

The present invention provides preparing and fitting prosthetics, particularly oral prosthetics. A protocol 100 for preparing and fitting a prosthetic in a prosthetic field of a patient using pre-surgical fiducial markers according to one aspect of the present invention is schematically illustrated in FIG. 1 and comprises the steps of: obtaining 10 pre-surgical physiologic data of the patient in a prosthetic field sufficient to create a base patient image 20 of the prosthetic field; determining or calculating 30 fiducial marker placement in physiologic structure within the prosthetic field of the patient which is to be unchanged by a planned surgery; placing 40 fiducial markers in the determined fiducial marker placement in physiologic structure within the prosthetic field which is to be unchanged by a planned surgery; obtaining 50 pre-surgical physiologic data of the patient in a prosthetic field with the fiducial markers in place sufficient to create a base patient image 60 of the prosthetic field with fiducial markers; performing surgery 70 altering physiologic structure of the patient within the prosthetic field; obtaining 80 post-surgical physiologic data of the patient in a prosthetic field with the fiducial markers in place sufficient to create a post-surgical patient image 90 of the prosthetic field with fiducial markers; manufacturing 110 and fitting 120 a final prosthetic based upon post-surgical patient image of the prosthetic field with fiducial markers.

As discussed above a prosthetic field within the meaning of this application is the area within the body, including related or adjacent tissue within which a prosthetic fits and operates. Further, the prosthetic field for oral prosthetics is the oral cavity and associated body structures, also generally called the patient's mouth.

The present invention is not limited to oral prosthetics, however this is one preferred implementation and a detailed discussion of this application will assist in understanding the scope and content of the present invention. The following discussion centers on an oral prosthetic within the protocol of the present invention.

### 10 Pre-Surgical Physiologic Data

The initial step of the protocol 100 is obtaining 10 pre-surgical physiologic data of the patient in a prosthetic field sufficient to create a base patient image or model 20 of the prosthetic field. It should be apparent to those of ordinary skill in the art that variations of the following procedures are possible to provide the full patient model 20. For example some current CBCT scanners can now detect tissue and facial recognition techniques are also improving. The integration of the following technology are described to obtain sufficient information for a complete and full patient model 20, and some of the technologies may yield redundant information and can be omitted.

The clinician can collect patient data using various tools and methodologies such as a Cone Beam CT scan. The Cone Beam CT scan uses Cone beam computed tomography (or CBCT), also referred to as C-arm CT, cone beam volume CT, flat panel CT or Digital Volume Tomography (DVT)) is a medical imaging technique consisting of X-ray computed tomography where the X-rays are divergent, forming a cone. CBCT is commonly used in treatment planning and diagnosis in implant dentistry as well as ENT, orthopedics, and interventional radiology (IR), among other things. CBCT scanners have many uses in dentistry, such as in the fields of oral surgery, endodontics and orthodontics.

During dental/orthodontic imaging, the CBCT scanner rotates around the patient's head, obtaining up to nearly 600 distinct images. Generally a single 200 degree rotation over the region of interest acquires a volumetric data set. The scanning software collects the data and reconstructs it, producing what is termed a “digital volume” composed of three-dimensional voxels of anatomical data that can then be manipulated and visualized with specialized software. In other words it forms a 3D model of the physiologic structure. CBCT shares many similarities with traditional (fan beam) CT, and CBCT has been described as the gold standard for imaging the oral and maxillofacial area and is well suited to form or assist in forming the base image or model 20 of the present invention.

Within the step of obtaining 10 pre-surgical physiologic data of the patient, the clinician will typically perform a patient examination and render a diagnosis of pathology if applicable, noting the chief concern and desires.

Further, within the step of obtaining 10 pre-surgical physiologic data of the patient the clinician may obtain a full set of photographic images of the patient's mouth. Within the meaning of this application a full set of photographic images of the patient's mouth includes a minimum of seven photographic views must be taken, which include: frontal smile, frontal retracted smile, smiling right quad, smiling left quad, upper jaw, lower jaw, left profile view.

In addition to scans and photographs, within the step of obtaining 10 pre-surgical physiologic data of the patient the clinician may obtain x-rays or radiographs of the patient's mouth.

Within the step of obtaining 10 pre-surgical physiologic data of the patient, the clinician will typically obtain measurements of “facial thirds” of the patient. A person's face is about one and a half times longer than its width. Measuring the facial thirds is measuring i) the length of the forehead hairline to a spot between the eyes, ii) the length between the eyes to the bottom of the nose, and iii) the length from the bottom of the nose to the bottom of the chin.

Within the step of obtaining 10 pre-surgical physiologic data of the patient, the clinician will typically perform an evaluation of the existing dentition of prosthesis as well.

An important and unique aspect of the protocol 100 of the present invention is within the step of obtaining 10 pre-surgical physiologic data of the patient, the clinician will document patient existing level of satisfaction/dis-satisfaction. This documentation is helpful in order to establish patient expectations. If patient is accepting the current smile and esthetics, the patient's file should so indicate. Similarly, patient dissatisfaction should be recorded and noted and the patient's existing level of satisfaction/dis-satisfaction can be helpful in designing an appropriate prosthetic and managing patient expectations for improved patient results. In other words the prosthetic may be designed to mimic the existing physiologic features that are satisfying to the patient or to minimize the areas of patient dissatisfaction. Patient outcome and satisfaction is greatly improved with these considerations.

The step of obtaining 10 pre-surgical physiologic data of the patient will have the clinician document the information. This will include having the clinician record impressions of arches and prosthetics with intraoral scanner (IOS) or in the analog version (physical impressions). The clinician will obtain digital (or analog) impressions of both upper and lower arches with proper muco-buccal fold extensions. For reference the muco-buccal fold is along the back wall of the mouth, the ridge of the oral mucosa that runs from the maxilla (superiorly) or the mandible (inferiorly) to the cheek.

The step of obtaining 10 pre-surgical physiologic data of the patient may have the clinician obtain a facial recognition scan of the patient, which is added to patient record. Traditional face recognition algorithms identify facial features by extracting landmarks, or features, from an image of the subject's face. For example, an algorithm may analyze the relative position, size, and/or shape of the eyes, nose, cheekbones, and jaw. The facial recognition scan may use a traditional algorithm and or a Three-dimensional face recognition technique. The Three-dimensional face recognition technique uses 3D sensors to capture information about the shape of a face. This information is then used to identify distinctive features on the surface of a face, such as the contour of the eye sockets, nose, and chin. One advantage of 3D face recognition is that it is not affected by changes in lighting. Three-dimensional data points from a face vastly improve the precision of face recognition. 3D-dimensional face recognition research is enabled by the development of sophisticated sensors that project structured light onto the face.

### Base Patient Image 20

As noted above the step of obtaining 10 pre-surgical physiologic data of the patient in a prosthetic field is sufficient to create a detailed base patient image or model 20 of the prosthetic field. The collection of scans and images and X-rays are combined to form a detailed base image or model 20 of the patient. The additional information, like patient satisfaction/dissatisfaction will supplement this image or model 20 and considered a part thereof.

### Fiducial Markers 35

FIGS. 2A-C are front elevational, side elevation and top plan views of a fiducial marker 35 used in the protocol of the present invention. The fiducial markers 35 can be made of a variety of radio-opaque bio-compatible materials as generally known in the art. The fiduciary markers 35 include a screw anchoring base for securing the fiduciary marker in a non-movable position. Additionally the faceted, non-symmetrical upper portion allows the markers 35 to be used for marking angular positioning of physiologic structure relative to the placed markers 35.

### Calculating 30 Fiducial Marker 35 Placement

The protocol 100 for preparing and fitting a prosthetic in a prosthetic field of a patient using pre-surgical fiducial markers 35 according to one aspect of the present invention includes the step of determining or calculating (at step 30) fiducial marker 35 placement in physiologic structure within the prosthetic field of the patient which is to be unchanged by a planned surgery. Following the creation of the base image or model 20 of the patient, an initial “Digital Implant Plan” is created for a proposed oral prosthetic.

Typically STL files associated with the initial prosthetic can be created that may be shipped to an off-site manufacturer or lab. The lab could also be in-house and is referenced herein only to define the technicians specializing in the prosthetic design and fiduciary marker 35 placement in the protocol 100, and the lab personnel will typically (but not necessarily) be different from the clinician/dentist.

STL is a file format native to the stereolithography CAD software created by 3D Systems. This file format is supported by many software packages and it is widely used for rapid prototyping, 3D printing and computer-aided manufacturing. STL files describe only the surface geometry of a three-dimensional object without any representation of color, texture or other common CAD model attributes. An STL file describes a raw, unstructured triangulated surface by the unit normal and vertices (ordered by the right-hand rule) of the triangles using a three-dimensional Cartesian coordinate system.

The lab will typically also plan a Surgical Guide in case the surgery will be guided. If not, lab will typically plan a trough guide for the surgical zone. A Surgical Guide provides the absolute directed surgical positional implant drilling and placement and is a guide that is affixed to the jawbone with fixation pins. The Surgical Guide typically replicates the exact surfaces of the patient's intraoral setting and assists the surgeon to drill implants into the bone with optimal accuracy. Upon placement on the patient's jaw, the surgical guide typically uses sleeves to help guide the surgical instruments and implant to the proper location Alternatively, different types of guide may be utilized, for example what is known as a Trough Guide may be used for those applications that will do the positional implant placement using manual positioning, however the exit screw hole positions here must exit through the trough guide.

The lab will also determine or calculate the placement of the fiducial markers 35 in physiologic structure within the prosthetic field of the patient which is to be unchanged by a planned surgery. The protocol 100 may implement fiducial markers 35 in different carriers or guides (collectively referenced herein as a placement ring). The lab will typically plan the deployment of the fiducial markers 35 which may be employed using various methods according to the specificity of the case. Individual fiducial markers 35 may be straight and angled with FIGS. 2A-C showing one representative shape. The fiducial markers 35 may be attached to a positioning placement ring; fiducial markers 35 may be attached to a part of the bone reduction guide, and fiducial markers 35 may be attached to a part of a prep guide to be used when teeth are being maintained.

The Lab can then create a preliminary prosthesis cad design which may be sent to the clinician/dentist for approval. This design will include/incorporate multi layers of data. The original patient data, with the new designed overlaid smile, implant positions, surgical guide(s), midline, and horizontal cant of the occlusal plane. Once design is approved by doctor, the initial or preliminary prosthetic gets milled or printed and completed by the lab and returned to clinician/doctor for the day of surgery while still on a puck which allows for final milling/machining as discussed below.

### Placing 40 Fiducial Markers 35

The protocol 100 for preparing and fitting a prosthetic in a prosthetic field of a patient using pre-surgical fiducial markers 35 according to one aspect of the present invention includes the step of placing 40 the fiducial markers 35, which is generally done the day of surgery of the patient.

The step of placing 40 the fiducial markers 35 preferably has an initial step of the clinician verifying the presence and accuracy of the initial prosthetic and guides and fiducial markers 35. This step is helpful to make sure all requisite materials are present prior to beginning.

The step of placing 40 the fiducial markers 35 may include an initial IOS scan of the patient prior to placement of the fiducial markers 35, which may identify changes in the patient physiology since the initial information was obtained and prior to implanting the fiducial markers 35. Any variances can be viewed and considered to see if alterations in the plan are required.

The step of placing 40 the fiducial markers 35 will typically use a placement ring with the fiducial markers 35 to accurately place the fiducial markers 35 in physiologic structure within the prosthetic field of the patient which is to be unchanged by a planned surgery. Fiducial markers 35 are specialized markers that are supplemented onto the prosthetic field as the key reference points for the patient's registration procedure. The use of fiducial markers 35 allows practitioners to correctly identify placement position for oral prostheses to ensure the correct fit as detailed herein. Fiducial markers 35 also make the transition from virtual model to a physical prosthesis easier and more accurate. The fiducial markers 35 allow the proper positioning of implants in a proper XYZ orientation as well as addressing angular orientations of the relevant elements.

Obtaining 50 Pre-Surgical Physiologic Data of the Patient in a Prosthetic Field with the Fiducial Markers

The protocol 100 for preparing and fitting a prosthetic in a prosthetic field of a patient using pre-surgical fiducial markers 35 according to one aspect of the present invention includes the step of obtaining 50 pre-surgical physiologic data of the patient in a prosthetic field with the fiducial markers 35 in place sufficient to create a base patient image or model 60 of the prosthetic field with fiducial markers 35. The step of obtaining 50 pre-surgical physiologic data of the patient in a prosthetic field with the fiducial markers 35 in place includes an IOS scan of the patient following placement of the fiducial markers 35 and prior to surgery.

Base Patient Image 60 of the Prosthetic Field with Fiducial Markers

The step of obtaining 50 pre-surgical physiologic data of the patient in a prosthetic field with the fiducial markers 35 in place is used to create a base patient image or model 60 of the prosthetic field with fiducial markers 35. Essentially this is an overlay of the prior patient images/models showing the added fiducial markers 35. This may also show changes in the physiology from the initial patient data which can be examined in case the clinician determines this warrants reconsideration of planned treatment.

### Surgery 70

The protocol 100 for preparing and fitting a prosthetic in a prosthetic field of a patient using pre-surgical fiducial markers 35 according to one aspect of the present invention includes the step of performing surgery 70 altering physiologic structure of the patient within the prosthetic field. The surgery may be guided or free-hand.

The surgery will install the implants needed for the oral prosthesis. A dental implant (also known as an endosseous implant or fixture) is a component of the oral prosthesis that interfaces with the bone of the jaw or skull to support the oral prosthesis such as a crown, bridge, denture, or facial prosthesis. The basis for modern dental implants is a biologic process called osseo-integration, in which materials such as titanium or zirconia form an intimate bond to bone. The implant fixture is first placed so that it is likely to osseo-integrate, then a dental prosthetic or oral prosthesis is added.

An abutment is a component of the oral prosthesis that couples the dental prosthetic (crown, bridge, denture) to the implant. It should be noted that an abutment is not necessarily parallel to the long axis of the implant.

Once implants are placed, the clinician will verify fiduciary markers 35 are still in place and non-mobile. For example, use of the PENGUIN® brand resonance frequency analysis for implant stability showing results >70 is preferred. In the unlikely event that the fiduciary markers 35 fail this stability test, then a new ground position or true position of the fiduciary markers 35 must be evaluated and used. Additionally, the reasons for the shift of the fiducial marker 35 position should be identified and evaluated.

Following the stability testing of the fiduciary markers 35, the clinicians will install the selected abutments, which may include multi-unit abutments. These are abutments that comes either straight or angled, and are attached to the implant body and torqued to specific strength. This abutment will fixate the components and prosthetic appliance to the implants. A disinfectant, antibacterial, and antifungal agent that reduces the incidence of bacterial contamination may be used around the threads of abutments.

### Obtaining 80 Post-Surgical Physiologic Data

The protocol 100 for preparing and fitting a prosthetic in a prosthetic field of a patient using pre-surgical fiducial markers 35 according to one aspect of the present invention includes the step of obtaining 80 post-surgical physiologic data of the patient in a prosthetic field with the fiducial markers 35 in place sufficient to create a post-surgical patient image or model 90 of the prosthetic field with fiducial markers 35 and implants with abutments.

This step can include placing titanium cylinders on each abutment and or fiducial marker 35 before scanning. The use of common cylinders can make it easier to align/adjust the actual placement and orientation of the implants/abutments for the final prosthetic. The step of obtaining 80 post-surgical physiologic data of the patient in a prosthetic field then includes an IOS scan all implant/cylinder positions. Alternatively Photogrammetry can also used for step 80, which is a method of recording the actual implant body positions in depth, pitch, rotation, and angulation, which is recorded by a camera type device. That data is converted to an STL file that can be used by the prosthetic design software. Following obtaining of the post-surgical data at 80 the location of the implants is precisely known, and the fiducial markers 35 may now be removed.

A Post-Surgical Patient Image 90 of the Prosthetic Field with Fiducial Markers 35

The step of obtaining 80 post-surgical physiologic data of the patient in a prosthetic field with the fiducial markers 35 in place sufficient to create a post-surgical patient image 90 of the prosthetic field with fiducial markers 35 and implants with abutments. This data may be sent to the cloud for data conversion. As with image 60 essentially this is an overlay of the prior patient images showing the fiducial markers 35 together with the implants. This will show changes in the physiology from the initial patient data. Further this image shows variations in the real world position and orientation of the implants from the original plan.

### Manufacturing 110 of a Final Prosthetic

The protocol 100 for preparing and fitting a prosthetic in a prosthetic field of a patient using pre-surgical fiducial markers according to one aspect of the present invention includes the step of manufacturing 110 of a final prosthesis.

Once data from the analysis of the image 90 is returned, usually within 30 minutes . . . the milling machine (or alternatively a 3D printer) is ready to start. The Prosthesis on the puck should already be in the milling machine in the predetermined position (i.e. the Puck is indexed for proper positioning). The manufacturing of the final prosthesis proceeds with tooling process to cut the tissue side and implant circumferential position holes (Process duration approx. 30-40 minutes. The milling of the final prosthesis is completed in the milling cycle and is retrieved from the puck. It is removed, cleared of all support pegs, polished and prepared for disinfection. Titanium base female components (if designed and used) are inserted and cemented into the implant positions within the proper implant channels. Once disinfected, it is returned to the doctor/assistant for “try-in” and first delivery.

### Fitting 120 Final Prosthesis

The protocol 100 for preparing and fitting a prosthetic in a prosthetic field of a patient using pre-surgical fiducial markers 35 according to one aspect of the present invention includes the step of fitting 120 of a final prosthesis. The clinician can screw in and verify the correct bite plane. Verification is completed by use of x-rays and photography. If acceptable, then the clinician may use gel impregnated foam bead with a disinfectant, antibacterial, and antifungal agent that reduces the incidence of bacterial contamination around within the screw channels of abutments and prosthetic retention channels, by placing 1-2 beads in the screw channels and seal access with proper colored composite.

Patient is often recalled post-surgery usually 1-3 days after surgery as indicated by the doctor. This Case is typically complete following this visit

### Follow-Up

The present protocol includes an optional 90-120 Day Review-Post Surgical Healing & Tissue Stability Review and Adjustment. In this step the patient returns, x-rays verify integrity of the prosthesis which must not be loose (or appropriate adjustments made). The clinician will adjust bite and make any necessary adjustments. Make sure to instruct on good and proper home hygiene. The prosthesis is removed, abutments are cleaned, and any hygiene instructions are again discussed. Another IOS scan of the tissue surface is created and uploaded to the current patient image. Photogrammetry can also be used in this step. The prosthetic is replaced into the patient's mouth after having applied an antibacterial protection layer disallowing the plaque and calculus from firmly adhering. The protection layer is applied to the abutments and prosthesis. This will begin the process of a “clean foundation” for the final rehabilitation. The base lab will upload the new tissue data and confirm proper stitching. Once all data is confirmed, if needed a new final fixed prosthesis is designed using the cad program of choice and the final material of choice. The MUA abutments are re-torqued using the specified manufacturer's recommendations. X-ray confirmation if necessary. Make any necessary adjustments to accommodate speech, lip posture, food trappings, etc. The protocol 100 should yield very minimal if any adjustments at this point. In fact, if any substantial adjustments necessary, then one would generally look back to where the data collection broke down.

### Alternative Protocol

The above methodology is to describe and walk through the aspects of the present invention for illustrative purposes. The protocol 100 may be streamlined with the omission of selective scanning and image or model creation. The scanning acquiring of digital information of fiduciary markers could be done simultaneously with the scanning of the anatomy. The basis is, when the first data capture of information is generated it is sent to lab. That data capture is of existing anatomy of patient by which the lab develops the prosthetic to be fabricated in relation to patient's anatomy. In later two data acquisition, the fiducial markers must be placed and the data acquired in relationship to said original anatomy. That placement then creates the foundation of data that will now have a relationship to the fiducial markers and data acquisition post-surgery. Again, it is important to note that it the anatomy of the patient completely altered during surgery. Thus the way the prosthetic that was developed to the original anatomy can be related to new anatomy is through the constant unchanging position of the fiducial markers pre and post-surgery as it relates to anatomy pre and post-surgery. However this constant position does not require a separated data acquisition of fiduciary markers on their own and the protocol 100 can be streamlined.

Further as noted above the present invention is described in connection with dental applications, which is a primary market for the technology. The present protocol has application in other fields, particularly in orthopedic and ophthalmological applications.

The above description is representative of the present invention but not restrictive thereof. The full scope of the present invention are set forth in the appended claims and equivalents thereto.

1. A protocol for preparing and fitting a prosthetic in a prosthetic field of a patient using pre-surgical fiducial markers comprising the steps of:
Obtaining pre-surgical physiologic data of the patient in a prosthetic field sufficient to create a base patient image of the prosthetic field;
Determining fiducial marker placement in physiologic structure within the prosthetic field of the patient which is to be unchanged by a planned surgery;
Placing fiducial markers in the determined fiducial marker placement in physiologic structure within the prosthetic field which is to be unchanged by a planned surgery;
Optionally obtaining pre-surgical physiologic data of the patient in a prosthetic field with the fiducial markers in place sufficient to create a base patient image of the prosthetic field with fiducial markers;
Performing surgery altering physiologic structure of the patient within the prosthetic field;
Obtaining post-surgical physiologic data of the patient in a prosthetic field with the fiducial markers in place sufficient to create a post-surgical patient image of the prosthetic field with fiducial markers;
Manufacturing a final prosthetic based upon post-surgical patient image of the prosthetic field with fiducial markers; and
Fitting the final prosthetic in the prosthetic field based upon post-surgical patient image of the prosthetic field with fiducial markers.

2. The protocol for preparing and fitting prosthetics in a prosthetic field of a patient according to claim 1, wherein the prosthetic is an oral prosthetic and the prosthetic field is the oral cavity of the patient.

3. The protocol for preparing and fitting prosthetics in a prosthetic field of a patient according to claim 2, wherein the step of obtaining pre-surgical physiologic data of the patient in a prosthetic field sufficient to create a base patient image includes data sufficient to establish frontal smile, frontal retracted smile, smiling right quad, smiling left quad, upper jaw, lower jaw, left profile view.

4. The protocol for preparing and fitting prosthetics in a prosthetic field of a patient according to claim 2, wherein the step of obtaining pre-surgical physiologic data of the patient includes digital dental information.

5. (canceled)

6. The protocol for preparing and fitting prosthetics in a prosthetic field of a patient according to claim 2, wherein the step of obtaining pre-surgical physiologic data of the patient includes facial recognition scans.

7. The protocol for preparing and fitting prosthetics in a prosthetic field of a patient according to claim 2, wherein the base patient image of the prosthetic field is used to design a preliminary oral prosthetic for the patient.

8. The protocol for preparing and fitting prosthetics in a prosthetic field of a patient according to claim 2, wherein the step of manufacturing the final prosthetic based upon post-surgical patient image of the prosthetic field with fiducial markers includes milling the preliminary oral prosthetic.

9. The protocol for preparing and fitting prosthetics in a prosthetic field of a patient according to claim 2, wherein the step of fitting the final prosthetic in the prosthetic field based upon post-surgical patient image includes waiting a set period of time for post-surgical healing and tissue stability before a final fitting.

10. The protocol for preparing and fitting prosthetics in a prosthetic field of a patient according to claim 2, wherein the step of performing surgery altering physiologic structure of the patient within the prosthetic field, includes installing implants in the oral cavity wherein the post-surgical patient image of the prosthetic field with fiducial markers includes images of the implants.

11. Registration images used for preparing and fitting a prosthetic in a prosthetic field of a patient using pre-surgical fiducial markers, the registration images being a composite of comprising:
A base patient image of a prosthetic field of the patient obtained with pre-surgical physiologic data of the patient in the prosthetic field;
A pre-surgical base patient image of the prosthetic field with fiducial markers, wherein fiducial markers in the determined fiducial marker placement in physiologic structure within the prosthetic field which is to be unchanged by a planned surgery, and the pre-surgical base patient image of the prosthetic field with fiducial markers is obtained with physiologic data of the patient in a prosthetic field with the fiducial markers in place; and
a post-surgical patient image of the prosthetic field with fiducial markers, whereby manufacturing of a final prosthetic is based upon post-surgical patient image of the prosthetic field with fiducial markers and fitting the final prosthetic in the prosthetic field is based upon post-surgical patient image of the prosthetic field with fiducial markers.

12. The registration images for preparing and fitting prosthetics in a prosthetic field of a patient according to claim 11, wherein the prosthetic is an oral prosthetic and the prosthetic field is the oral cavity of the patient.

13. The registration images for preparing and fitting prosthetics in a prosthetic field of a patient according to claim 12, wherein the base patient image is formed from data sufficient to establish frontal smile, frontal retracted smile, smiling right quad, smiling left quad, upper jaw, lower jaw, left profile view.

14. The registration images for preparing and fitting prosthetics in a prosthetic field of a patient according to claim 12, wherein at least one of the images are formed from data including digital dental information.

15. The registration images for preparing and fitting prosthetics in a prosthetic field of a patient according to claim 12, wherein at least one of the images are formed from data including facial recognition scans.

16. A protocol for preparing and fitting an oral prosthetic in a patient using pre-surgical fiducial markers comprising the steps of:
Obtaining pre-surgical physiologic data of the patient in a prosthetic field sufficient to create a base patient image of the prosthetic field;
Determining fiducial marker placement in physiologic structure within the prosthetic field of the patient which is to be unchanged by a planned surgery;
Placing fiducial markers in the determined fiducial marker placement in physiologic structure within the prosthetic field which is to be unchanged by a planned surgery;
Optionally obtaining pre-surgical physiologic data of the patient in a prosthetic field with the fiducial markers in place sufficient to create a base patient image of the prosthetic field with fiducial markers;
Performing surgery altering physiologic structure of the patient within the prosthetic field;
Obtaining post-surgical physiologic data of the patient in a prosthetic field with the fiducial markers in place sufficient to create a post-surgical patient image of the prosthetic field with fiducial markers;
Manufacturing a final prosthetic based upon post-surgical patient image of the prosthetic field with fiducial markers; and
Fitting the final prosthetic in the prosthetic field based upon post-surgical patient image of the prosthetic field with fiducial markers.

17. The protocol for preparing and fitting an oral prosthetic in a patient using pre-surgical fiducial markers according to claim 16 wherein the base patient image is formed from data sufficient to establish frontal smile, frontal retracted smile, smiling right quad, smiling left quad, upper jaw, lower jaw, left profile view.

18. The protocol for preparing and fitting an oral prosthetic in a patient using pre-surgical fiducial markers according to claim 16 wherein the base patient image is formed from data including digital dental information.

19. The protocol for preparing and fitting an oral prosthetic in a patient using pre-surgical fiducial markers according to claim 16 wherein the base patient image is formed from data including facial recognition scans.

20. The protocol for preparing and fitting an oral prosthetic in a patient using pre-surgical fiducial markers according to claim 16 wherein the step of performing surgery altering physiologic structure of the patient within the prosthetic field, includes installing implants in the oral cavity wherein the post-surgical patient image of the prosthetic field with fiducial markers includes images of the implants.
